(19) United States
(12) Patent Application Publication
(10) Pub. No.: US 2024/0415462 A1
(43) Pub. Date: Dec. 19, 2024

(54) BIOPOTENTIAL MEASUREMENT DEVICE, INFORMATION PROCESSING APPARATUS, AND BIOPOTENTIAL MEASUREMENT METHOD

(71) Applicant: SONY GROUP CORPORATION, TOKYO (JP)

(72) Inventor: KAZUNARI YOSHIFUJI, TOKYO (JP)

(21) Appl. No.: 18/704,602

(22) PCT Filed: Oct. 24, 2022
(86) PCT No.: PCT/JP2022/039461

§ 371 Date: Apr. 25, 2024

(30) Foreign Application Priority Data
Nov. 5, 2021 (JP) 2021-180768

(51) Int. Cl. A61B 5/00; A61B 5/25; A61B 5/304

(52) U.S. Cl. CPC A61B 5/7203; A61B 5/25; A61B 5/304

(57) ABSTRACT

The present technology relates to a biopotential measurement device, an information processing apparatus, and a biopotential measurement method capable of improving input-referred noise performance of an analog front end.

The biopotential measurement device according to an aspect of the present technology sets a setting value of an offset potential to be applied to an input of an amplifier on the basis of a signal level of a digital signal after AD conversion and switch a connected state and a separated state of a switch. The switch is provided between a DA converter that converts a digital signal indicating a setting value into an analog signal and that generates an analog potential and a potential holder that holds the analog potential generated by the DA converter and that applies the held analog potential to the input of the amplifier. The present technology can be applied to wearable devices.

### TECHNICAL FIELD

The present technology relates to a biopotential measurement device, an information processing device, and a biopotential measurement method, and more particularly to a biopotential measurement device, an information processing device, and a biopotential measurement method capable of improving input-referred noise performance of an amplifier.

### BACKGROUND ART

In recent years, wearable devices capable of measuring biological signals such as pulse waves and electrocardiograms in daily life are commercially available. By measuring biological signals, it is possible to provide new experience and applications for users.

It is possible to measure electroencephalograms (EEGs) by using substantially the same method as a method for measuring biological signals used for measuring electrocardiograms and electromyograms, and wearable devices capable of measuring EEGs in daily life have also started to appear on the market. For example, a device that measures an EEG of a user during sleep and that determines a state of the sleep is commercially available.

Non-Patent Document 1 and Non-Patent Document 2 disclose techniques capable of measuring biological signals in a form in which a dynamic range and resolution are secured while using an analog digital (AD) converter with a low bit depth.

### CITATION LIST

### Non-Patent Document

Non-Patent Document 1: Catherine J. Poirier, “Hardware Implementation of an Adaptive Data Acquisition System for Real-World EEG,” 2018 40th Annual International Conference of the IEEE Engineering in Medicine and Biology Society (EMBC).
Non-Patent Document 2: Lianxi Liu, “A Robust Bio-IA With Digintally Controlled DC-Servo Loop and Improved Pseudo Resistor,” IEEE Transactions on circuits and systems II: express briefs, vol. 67, no. 3, March 2020.

### SUMMARY OF THE INVENTION

### Problems to be Solved by the Invention

In the techniques disclosed in Non-Patent Document 1 and Non-Patent Document 2, large variations in a level of an input signal to be subjected to AD conversion are canceled by inputting an offset potential of a DC component to an amplifier, in order to secure a dynamic range of an AD converter. The AD converter performs AD conversion on the signal for which variations in the level have been canceled in the amplifier.

Normally, a digital analog (DA) converter is used to input an offset potential to an amplifier. The DA converter is a circuit that generates an analog potential in response to specification of a digital value.

In such a configuration, since a noise component of a DA converter is superimposed upon an input signal of an amplifier, input-referred noise performance (input refer noise) of the amplifier in a period in which an offset potential is applied deteriorates, which results in deterioration of resolution of an EEG acquisition level of an entire system.

The present technology has been made in view of such circumstances and aims to improve input-referred noise performance of an amplifier.

### Solutions to Problems

A biopotential measurement device according to a first aspect of the present technology includes an amplifier that amplifies a potential that is a difference between a reference potential and a potential measured by an electrode attached to a measurement target position of a biopotential, an AD converter that converts an analog signal according to the potential amplified by the amplifier into a digital signal, a calculation unit that sets a setting value of an offset potential to be applied to an input of the amplifier on a basis of a signal level of the digital signal after AD conversion, a DA converter that converts a digital signal indicating the setting value into an analog signal and that generates an analog potential, a potential holder that holds the analog potential generated by the DA converter and that applies the held analog potential to the input of the amplifier, a switch provided between the DA converter and the potential holder, and a controller that switches a connected state and a separated state of the switch.

An information processing apparatus according to a second aspect of the present technology includes a biopotential measurement device including an amplifier that amplifies a potential that is a difference between a reference potential and a potential measured by an electrode attached to a measurement target position of a biopotential, an AD converter that converts an analog signal according to the potential amplified by the amplifier into a digital signal, a calculation unit that sets a setting value of an offset potential to be applied to an input of the amplifier on a basis of a signal level of the digital signal after AD conversion, a DA converter that converts a digital signal indicating the setting value into an analog signal and that generates an analog potential, a potential holder that holds the analog potential generated by the DA converter and that applies the held analog potential to the input of the amplifier, a switch provided between the DA converter and the potential holder, and a controller that switches a connected state and a separated state of the switch.

In the present technology, a setting value of an offset potential to be applied to an input of an amplifier is set on the basis of a signal level of a digital signal after AD conversion, and a connected state and a separated state of a switch are switched.

### BRIEF DESCRIPTION OF DRAWINGS

FIG. 1 is a diagram illustrating an example of an apparatus including a biosensor module to which the present technology is applied.

FIG. 2 is a diagram illustrating a configuration example of the biosensor module.

FIG. 3 is a diagram illustrating an example of changes in an offset potential.

FIG. 4 is a flowchart illustrating a process for switching an offset potential performed by the biosensor module.

FIG. 5 is a diagram illustrating a timing chart regarding switching of the offset potential.

FIG. 6 is a diagram illustrating another configuration example of the biosensor module.

FIG. 7 is another diagram illustrating a timing chart regarding switching of the offset potential.

### MODE FOR CARRYING OUT THE INVENTION

A mode for carrying out the present technology will be described hereinafter. The description will be given in the following order.
1. Measurement of EEG
2. Configuration of Biosensor Module
3. Operation of Biosensor Module
4. Modification
5. Others

### <1. Measurement of EEG>

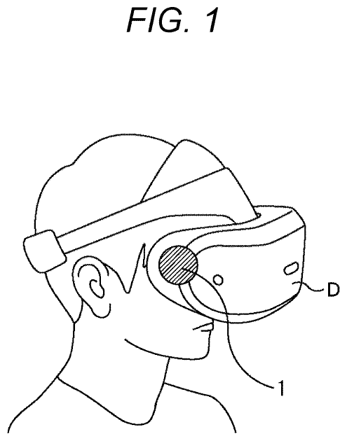

FIG. 1 is a diagram illustrating an example of an apparatus including a biosensor module according to an embodiment of the present technology.

A wearable device D illustrated in FIG. 1 is an information processing apparatus used when worn on the head. The wearable device D is used to watch video content, play a game, and for other purposes.

As schematically indicated by a hatched circle, a biosensor module 1 is mounted on the wearable device D. The biosensor module 1 measures a user's EEG at a predetermined timing such as during watching of video content or during playing of a game. The biosensor module 1 is not limited to a position indicated by the circle, and may be mounted at another position in the wearable device D.

Here, the measurement of an EEG will be described.

EEG signals are classified into θ waves (4 to 8 Hz), a waves (8 to 12 Hz), and B waves (12 Hz or higher) on the basis of frequency. Amplitude of an EEG signal when an a wave is dominant is about tens of μV.

As performance required for an electric circuit used to measure EEGs, it is specified by a standard that a frequency band of an analog signal be 0.5 Hz to 100 Hz and a value of input-referred noise be 1 μVrms or smaller in the frequency range. Input-referred noise performance is synonymous with resolution of a circuit. The smaller the value of the input-referred noise, the higher the performance.

It is difficult to achieve performance in which the input-referred noise is 1 μVrms or smaller in the frequency band of 0.5 Hz to 100 Hz unless parts of each of components are appropriately selected.

In addition, since EEG signals are weak, configuration of a first stage of the circuit needs to be a high-impedance configuration in order to represent an EEG as a signal. There are many circuits in which the configuration of the first stage has an impedance of 1 GΩ or higher.

Furthermore, a range of potential of EEG signals is very wide. In addition, the potential of EEG signals varies in a range of several hundred mV due to body movement or the like.

In general, in a medical electroencephalograph, impedance between an electrode and a living body is lowered and a phenomenon called “polarization”, which occurs at an interface between the electrode and the living body, is prevented by using a dedicated paste, gel, or the like. Polarization is a phenomenon caused by a chemical reaction at an interface, and corresponds to presence of a direct-current battery. A polarization voltage depends on a material of an electrode. The polarization voltage reaches several tens of mV and also fluctuates due to body motion.

In the case of various wearable devices including the wearable device D, a dry electrode is used to ensure comfortable wearability. That is, since a paste or gel for EEG measurement is not used, polarization tends to increase. A range of an input level of an EEG measurement circuit, therefore, is desirably wide, and a range of about ±300 mV is required.

As described above, the value of the input-referred noise required in the EEG measurement circuit is 1 μVrms. In a case where an AD converter converts a signal on this μV level into a digital signal, necessary resolution of the AD converter is provisionally set as resolution represented by the following Expression 1.

$$1 μ V \div 16 = 62.5 nV \qquad ( 1 )$$

A required bit depth of the AD converter at this time is obtained as 24 bits by the following Expressions 2 and 3 when a required dynamic range is a range of ±300 mV.

$$300 m V \times 2 \div 62.5 nV = 9600000 gradations \qquad ( 2 )$$

$$log 2 ( 9600000 ) = 23.19 \qquad ( 3 )$$

Normally, the lower the bit depth of the AD converter, the more the options for implementation. For example, in a case where an analog circuit and an AD converter are mounted on one semiconductor chip, selection of a 12-bit AD converter or a 16-bit AD converter can expand options of a semiconductor process, rather than selection of a 24-bit AD converter.

In order to lower the required bit depth of the AD converter, it is substantially difficult to lower the resolution, and it is therefore necessary to make the dynamic range of the input level narrower than the range of ±300 mV.

The biosensor module 1 mounted on the wearable device D is a biopotential measurement circuit that achieves low cost and low noise while securing a wide dynamic range and high resolution.

Note that a wearable device on which the biosensor module 1 is mounted is not limited to the wearable device D worn on the head. The biosensor module 1 can be mounted on a wearable device attached to other parts such as a wrist, a foot, or a torso.

In addition, biological signals measured by the biosensor module 1 are not limited to EEG signals. The biosensor module 1 can be used to measure signals representing various biological reactions appearing as changes in potential, such as electrocardiograms and electromyograms.

### <2. Configuration of Biosensor Module>

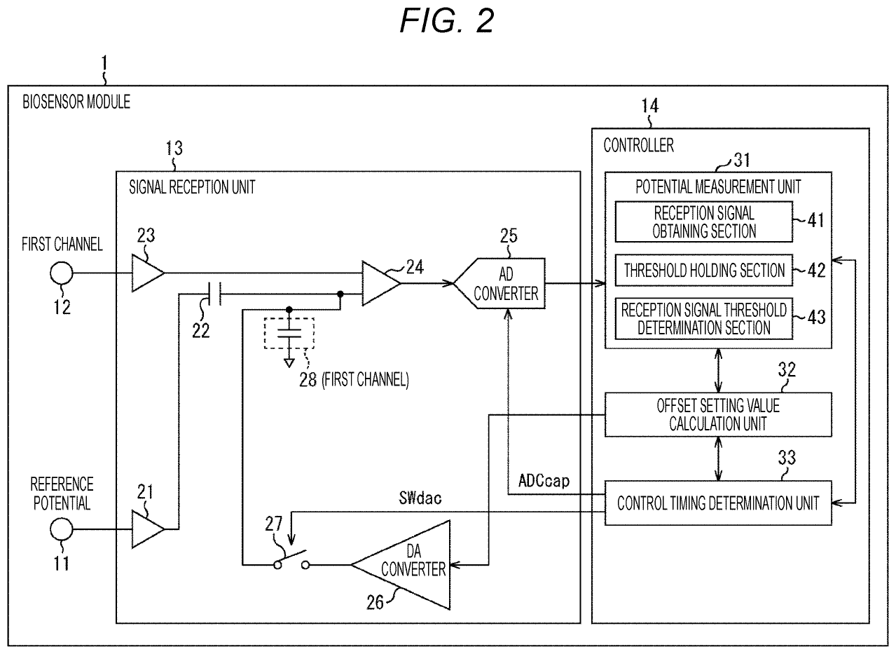

FIG. 2 is a diagram illustrating a configuration example of the biosensor module 1.

The biosensor module 1 includes a bioelectrode 11, a bioelectrode 12, a signal reception unit 13, and a controller 14. The biosensor module 1 is a biopotential measurement device used to measure biological signals such as EEG signals.

The signal reception unit 13 includes a buffer 21, a capacitor 22, a buffer 23, a differential amplifier 24, an AD converter 25, a DA converter 26, a switch 27, and a capacitor 28. As described later, an analog signal output from the DA converter 26 is supplied to the capacitor 28 via the switch 27. The capacitor 28 functions as a potential holder that holds a potential according to the analog signal output from the DA converter 26. The capacitor 28 will be described hereinafter as a potential holder 28 as appropriate.

A signal of a reference potential obtained by the bioelectrode 11 attached to a body surface is supplied to the buffer 21. The buffer 21 performs impedance conversion (current enhancement) on the signal obtained by the bioelectrode 11 and outputs the signal. The signal output from the buffer 21 is supplied to the differential amplifier 24 via the capacitor 22.

A signal of a certain potential obtained by the bioelectrode 12 attached to the body surface at a measurement target position is supplied to the buffer 23 as a signal of a first channel. The buffer 23 performs impedance conversion (current enhancement) on the signal obtained by the bioelectrode 12 and outputs the signal to the differential amplifier 24.

The differential amplifier 24 amplifies a potential that is a difference between the potential at the measurement target position represented by the signal supplied from the buffer 23 and the reference potential, which is the potential held by the potential holder 28 and applied as the offset potential, and outputs a signal according to the amplified potential. The signal output from the differential amplifier 24 is supplied to the AD converter 25.

The AD converter 25 performs AD conversion on the signal supplied from the differential amplifier 24 and outputs a signal (digital signal) obtained through the AD conversion at a certain cycle in accordance with an AD conversion timing signal ADCcap supplied from a control timing determination unit 33 of the controller 14. As described later, a signal after the AD conversion in a period in which the AD conversion timing signal ADCcap is “H”, for example, is output from the AD converter 25. The signal output from the AD converter 25 is supplied to a potential measurement unit 31 of the controller 14.

The DA converter 26 performs DA conversion on a signal supplied from an offset setting value calculation unit 32 of the controller 14 and outputs a potential (analog signal) generated through the DA conversion. A digital signal indicating a value (offset setting value) of an offset potential set by the offset setting value calculation unit 32 is supplied from the offset setting value calculation unit 32. The signal output from the DA converter 26 is supplied to the switch 27. As described above, the DA converter 26 is a circuit or a functional block that generates an analog potential in response specification of the offset setting value, which is a digital value, by the offset setting value calculation unit 32.

The switch 27 switches on and off in accordance with a switching signal SWdac supplied from the control timing determination unit 33. As described later, the switch 27 turns on in a period different from one in which the AD converter 25 supplies a signal after the AD conversion to the potential measurement unit 31 in response to the AD conversion timing signal ADCcap supplied from the control timing determination unit 33 to the AD converter 25 becoming “H”. In a case where the switch 27 is on, a potential generated by the DA converter 26 is supplied to the potential holder 28.

The potential holder 28 holds the potential generated by the DA converter 26 via the switch 27. The potential held by the potential holder 28 is applied to the reference potential as an offset potential.

The potential generated by the DA converter 26 (a potential corresponding to a signal subjected to DA conversion output from the DA converter 26) is held as a charge by the potential holder 28. When the potential generated by the DA converter 26 is updated, the switch 27 turns on. In addition, after the potential generated by the DA converter 26 is updated, the switch 27 turns off.

As a result, it becomes possible to maintain a state in which the offset potential is applied to the differential amplifier 24 even in a state in which a connection from the DA converter 26 is off.

The DA converter 26 is a circuit or a functional block that generates an analog potential from any digital value and mainly generates noise caused by thermal noise. In particular, the larger the potential generated by the DA converter 26, that is, the larger the potential applied to the differential amplifier 24 as the offset potential, the larger the noise generated by the DA converter 26.

Since the differential amplifier 24 and the DA converter 26 can be appropriately disconnected from each other by the switch 27 and a desired potential can be held by the potential holder 28, an effect of noise caused by the DA converter 26 can be eliminated even in a case where a desired offset potential is continuously applied. By turning off the switch 27 during a period in which the signal after the AD conversion is supplied from the AD converter 25 to the potential measurement unit 31, it is possible to prevent the effect of noise caused by the DA converter 26 upon a result of the AD conversion performed by the AD converter 25.

The controller 14 includes the potential measurement unit 31, the offset setting value calculation unit 32, and the control timing determination unit 33.

The potential measuring unit 31 includes a reception signal obtaining section 41, a threshold holding section 42, and a reception signal threshold determination section 43.

The reception signal obtaining section 41 receives a signal output from the AD converter 25 and obtains a digital signal value of an output signal of the differential amplifier 24. The digital signal value obtained by the reception signal obtaining section 41 is appropriately output to the outside of the biosensor module 1 as a measurement result in the biosensor module 1. The signal received by the reception signal obtaining section 41 is also output to the reception signal threshold determination section 43 and the like.

The threshold holding section 42 holds a threshold used to make a determination as to offset potential control. Two thresholds, namely an offset control threshold Th-H and an offset control threshold Th-L, which are thresholds used to make a determination as to the offset potential control, are set in advance. The two thresholds, namely the offset control threshold Th-H and the offset control threshold Th-L, held by the threshold holding section 42 are used by the reception signal threshold determination section 43.

The reception signal threshold determination section 43 compares a digital signal value Vadc of a reception signal with the thresholds held by the threshold holding section 42 and outputs a comparison result to the offset setting value calculation unit 32.

The offset setting value calculation unit 32 calculates an offset potential to be applied to the differential amplifier 24 on the basis of the comparison result obtained by the reception signal threshold determination section 43 and outputs a signal indicating an offset setting value to the DA converter 26.

The control timing determination unit 33 outputs the AD conversion timing signal ADCcap to the AD converter 25 and the switching signal SWdac to the switch 27. The control timing determination unit 33 controls an AD conversion timing of the AD converter 25 and the connected state and separated state (ON/OFF) of the switch 27. The control timing determination unit 33 controls both timings.

As described above, for example, the control timing determination unit 33 generates the switching signal SWdac such that the switch 27 turns on at a timing different from the AD conversion timing signal ADCcap. In addition, the control timing determination unit 33 controls the switch 27 such that the switch 27 turns off after the potential held by the potential holder 28 is updated.

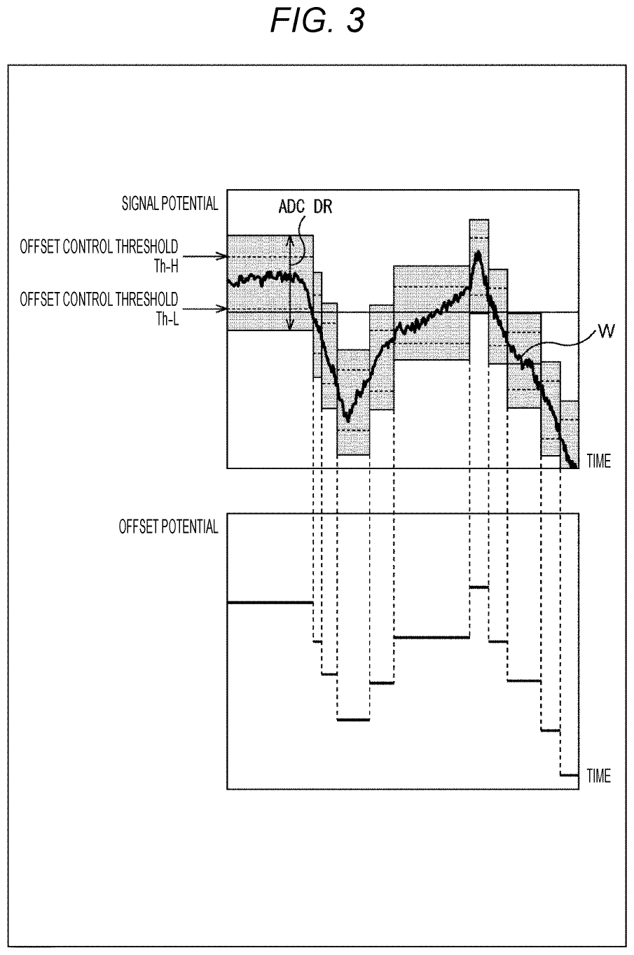

FIG. 3 is a diagram illustrating an example of changes in an offset potential.

A waveform W illustrated in an upper part of FIG. 3 indicates changes in potential of a signal input to the AD converter 25. A vertical axis in the upper part of FIG. 3 represents the potential of the input signal, and a horizontal axis represents time. A range indicated by a vertical arrow is a potential range (dynamic range) of the input signal that can be subjected to the AD conversion performed by the AD converter 25.

A lower part of FIG. 3 illustrates changes in the offset potential generated by the DA converter 26. A vertical axis in the lower part of FIG. 3 represents the offset potential, and a horizontal axis represents time.

As illustrated in FIG. 3, in a case where the potential of the input signal falls below the offset control threshold Th-L, a potential lower than a previous potential is set as a new offset potential. In response to an update of the offset potential, the dynamic range of the AD converter 25 is switched to a lower range.

Similarly, in a case where the potential of the input signal exceeds the offset control threshold Th-H, a potential higher than a previous potential is set as a new offset potential. In response to an update of the offset potential, the dynamic range of the AD converter 25 is switched to a higher range.

The threshold holding section 42 holds the offset control threshold Th-H and the offset control threshold Th-L used for the comparison with the potential of the input signal. The offset control threshold Th-H is a threshold that is set within the dynamic range and that is an upper limit of the potential of the input signal. In addition, the offset control threshold Th-H is a threshold that is set within the dynamic range and that is a lower limit of the potential of the input signal.

### <3. Operation of Biosensor Module>

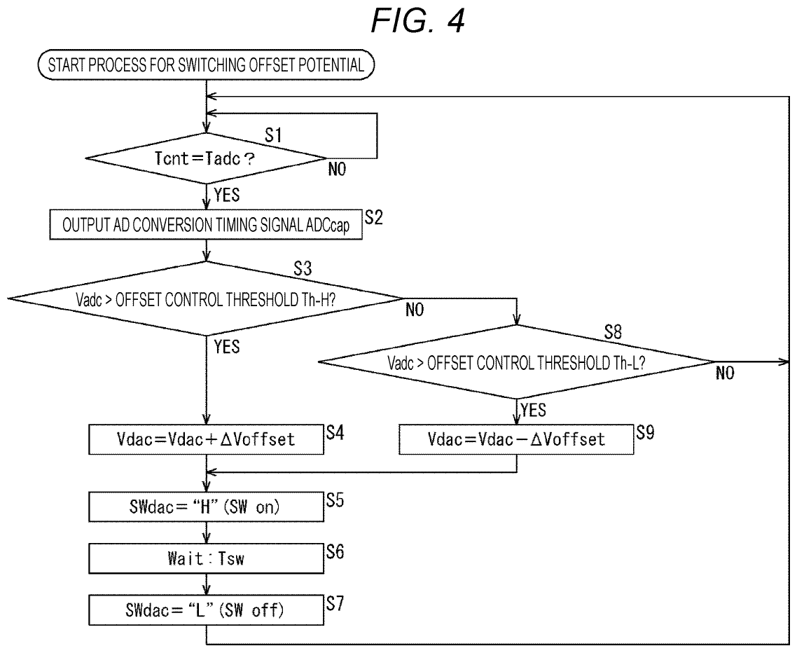

Here, a process for switching an offset potential performed by the biosensor module 1 will be described with reference to a flowchart of FIG. 4.

In step S1, the control timing determination unit 33 determines whether or not a counter value Tcnt managed thereby is equal to a value Tadc, and waits until determining that these values are equal. For example, the value Tadc is an AD conversion sampling frequency, and a value Tadc corresponding to a certain period of time such as 1 ms is set in advance.

In a case where the control timing determination unit 33 determines in step S1 that the counter value Tcnt is equal to the value Tadc since the certain period of time such as 1 ms has elapsed, the control timing determination unit 33 outputs the AD conversion timing signal ADCcap to the AD converter 25 in step S2. As the AD conversion timing signal ADCcap is output from the control timing determination unit 33, the signal after the AD conversion is output from the AD converter 25 to the potential measurement unit 31 and obtained as a reception signal.

In step S3, the reception signal threshold determination section 43 determines whether or not a digital signal value Vadc of the reception signal is larger than the offset control threshold Th-H.

In a case where the reception signal threshold determination section 43 determines in step S3 that the digital signal value Vadc is larger than the offset control threshold Th-H, the offset setting value calculation unit 32 calculates, in step S4, a new offset setting value Vdac by adding an offset potential ΔVoffset to a current offset setting value Vdac. For example, a potential according to how greatly the digital signal value Vadc exceeds the offset control threshold Th-H is added as the offset potential ΔVoffset. The offset setting value calculation unit 32 outputs a signal indicating the newly obtained offset setting value Vdac to the DA converter 26.

In step S5, the control timing determination unit 33 outputs a switching signal SWdac “H” to the switch 27. The switch 27 turns on, and an offset potential according to the new offset setting value Vdac generated by the DA converter 26 is supplied to the potential holder 28.

In step S6, the control timing determination unit 33 waits until a period of time Tsw, which is a certain period of time, elapses.

When the certain period of time Tsw has elapsed, the control timing determination unit 33 outputs, in step S7, the switching signal SWdac “L” to the switch 27. The switch 27 turns off, and the potential holder 28 holds a potential according to the new offset setting value Vdac. In addition, an offset potential according to the new offset setting value Vdac is applied to the differential amplifier 24.

In a case where the reception signal threshold determination section 43 determines in step S3 that the digital signal value Vadc is not larger than the offset control threshold Th-H, the reception signal threshold determination section 43 determines, in step S8, whether or not the digital signal value Vadc is smaller than the offset control threshold Th-L.

In a case where the reception signal threshold determination section 43 determines in step S8 that the digital signal value Vadc is smaller than the offset control threshold Th-L, the offset setting value calculation unit 32 calculates, in step S9, a new offset setting value Vdac by subtracting an offset potential ΔVoffset from the current offset setting value Vdac. The offset setting value calculation unit 32 outputs a signal indicating the newly obtained offset setting value Vdac to the DA converter 26.

After the new offset setting value Vdac is set in step S9, the process proceeds to step S5, and processing similar to the above-described processing is performed. The potential holder 28 is in a state of holding the potential according to the new offset setting value Vdac, and an offset potential according to the new offset setting value Vdac is applied to the differential amplifier 24.

After the switch 27 turns off in step S7, or in a case where the reception signal threshold determination section 43 determines in step S8 that the digital signal value Vadc is not smaller than the offset control threshold Th-L, the process returns to step S1, and the above process is repeated.

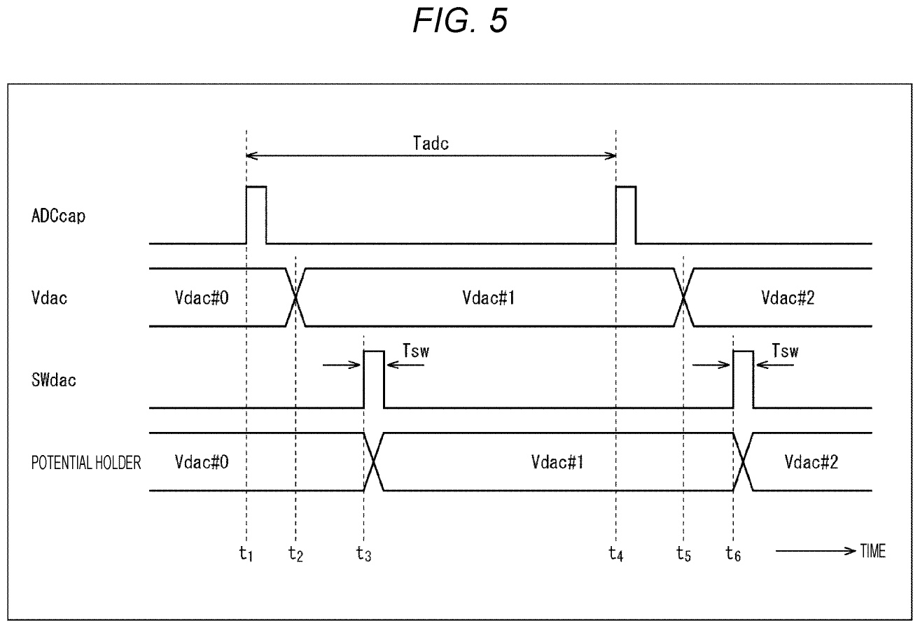

FIG. 5 is a diagram illustrating a timing chart regarding switching of the offset potential.

As illustrated in an uppermost part of FIG. 5, the AD conversion timing signal ADCcap becomes “H” at a cycle according to the count value Tadc. The AD conversion unit 25 performs AD conversion when the AD conversion timing signal ADCcap is “H”. In addition, the reception signal threshold determination section 43 compares the digital signal value Vadc of the reception signal with the thresholds. The offset setting value is updated and the switch 27 is turned on or off as appropriate on the basis of a result of the comparison.

In the example illustrated in FIG. 5, the AD conversion timing signal ADCcap becomes “H” at a time t₁, and as illustrated in a second part, the offset setting value Vdac according to the digital signal value Vadc of the received signal is updated from an offset setting value Vdac #0 to an offset setting value Vdac #1 at a time t₂. The DA converter 26 performs DA conversion on a digital signal according to the offset setting value Vdac #1 and outputs a signal obtained through the DA conversion.

As illustrated in a third part, at a time t₃ after the offset setting value Vdac is updated, the switching signal SWdac becomes “H”, and the switch 27 turns on. In addition, as illustrated in a fourth part, immediately after the switch 27 turns on, the potential held by the potential holder 28 is updated from the value Vdac #0 to the value Vdac #1. After the potential held by the potential holder 28 is updated, the switching signal SWdac becomes “L”, and the switch 27 turns off. The switching signal SWdac becomes “H” for the period of time Tsw.

A similar process is performed after a time t₄. That is, the AD conversion timing signal ADCcap becomes “H” at the time t₄, and the offset setting value is updated from the offset setting value Vdac #1 to an offset setting value Vdac #2 at a time t₅.

At a time to, the switching signal SWdac becomes “H”, and immediately after that, the potential held by the potential holder 28 is updated from the value Vdac #1 to the value Vdac #2, and the switch 27 turns off.

As illustrated in FIG. 5, since the AD conversion timing signal ADCcap becomes “H” at a certain cycle, the AD converter 25 outputs a result of the AD conversion, that is, obtains a digital signal value, at the certain cycle. In addition, in response to the obtaining of a digital signal value at the certain cycle, the switch 27 is also controlled at the certain cycle using the switching signal SWdac.

As described above, by changing the dynamic range of the input of the AD converter 25 in accordance with a signal level of a reception signal, a wide dynamic range and high resolution can be achieved. In addition, choices of the AD converter 25 can be increased, and cost can be reduced.

Furthermore, the offset potential applied to change the dynamic range is updated in a period different from one in which a signal after AD conversion is output from the AD converter 25, and after the offset potential is updated, the AD converter 25 and the DA converter 26 are disconnected. This makes it possible to prevent noise caused by the DA converter 26 from affecting the input-referred noise of the amplifier.

That is, with the biosensor module 1, it is possible to achieve cost reduction and noise reduction while securing a wide dynamic range and high resolution.

### <4. Modification>

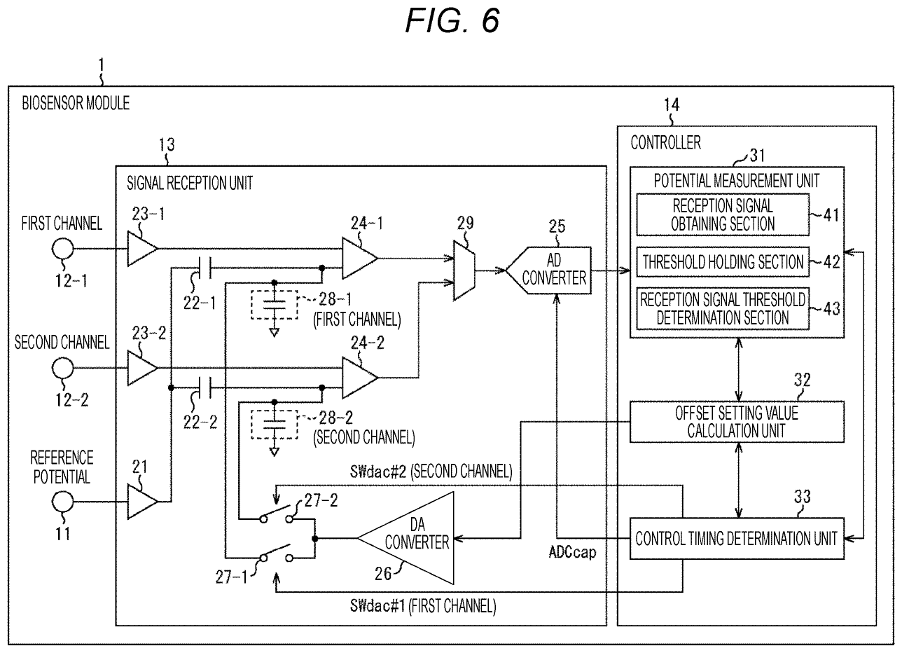

FIG. 6 is a diagram illustrating another configuration example of the biosensor module 1.

Among components illustrated in FIG. 6, the same components as those described with reference to FIG. 2 are given the same reference numerals. Redundant description is omitted as appropriate. The configuration illustrated in FIG. 6 is different from that described with reference to FIG. 2 in that there are a plurality of measurement target points, a signal obtained by a bioelectrode 12-1 is supplied to the signal reception unit 13 as a signal of a first channel, and a signal obtained by a biological electrode 12-2 is supplied to the signal reception unit 13 as a signal of a second channel.

The signal reception unit 13 is provided with a configuration for processing the signal of the second channel in addition to a configuration for processing the signal of the first channel. A capacitor 22-1, a buffer 23-1, a differential amplifier 24-1, a switch 27-1, and a potential holder 28-1 are provided for the signal of the first channel, and a capacitor 22-2, a buffer 23-2, a differential amplifier 24-2, a switch 27-2, and a potential holder 28-2 are provided for the signal of the second channel.

A signal output from the buffer 21 is supplied to the differential amplifier 24-1 and the differential amplifier 24-2 through the capacitor 22-1 and the capacitor 22-2, respectively.

The buffer 23-1 amplifies the signal supplied from the bioelectrode 12-1 and outputs the amplified signal to the differential amplifier 24-1.

The differential amplifier 24-1 amplifies a potential that is a difference between a potential at a measurement target position represented by the signal supplied from the buffer 23-1 and a reference potential, which is a potential held by the potential holder 28-1 and applied as an offset potential, and outputs a signal according to the amplified potential. The signal output from the differential amplifier 24-1 is supplied to a selector 29.

The buffer 23-1 amplifies the signal supplied from the bioelectrode 12-2 and outputs the amplified signal to the differential amplifier 24-2.

The differential amplifier 24-2 amplifies a potential that is a difference between a potential at a measurement target position represented by the signal supplied from the buffer 23-2 and a reference potential, which is a potential held by the potential holder 28-2 and applied as an offset potential, and outputs a signal according to the amplified potential. The signal output from the differential amplifier 24-2 is supplied to the selector 29.

The selector 29 selects the signal of the first channel supplied from the differential amplifier 24-1 or the signal of the second channel supplied from the differential amplifier 24-2 and outputs the selected signal to the AD converter 25. The output of the selector 29 is switched at a certain timing.

The switch 27-1 switches on and off in accordance with a switching signal SWdac #1 supplied from the control timing determination unit 33.

The potential holder 28-1 holds a potential according to a signal output from the DA converter 26 and supplied through the switch 27-1.

The switch 27-2 switches on and off in accordance with a switching signal SWdac #2 supplied from the control timing determination unit 33.

The potential holder 28-2 holds a potential according to a signal output from the DA converter 26 and supplied through the switch 27-2.

The control timing determination unit 33 outputs the switching signal SWdac #1 to the switch unit 27-1 to control on and off of the switch 27-1. In addition, the control timing determination unit 33 outputs the switching signal SWdac #2 to the switch unit 27-2 to control on and off of the switch 27-2. The control timing determination unit 33 controls the switch 27-1 and the switch 27-2 such that a connected state is established in different periods.

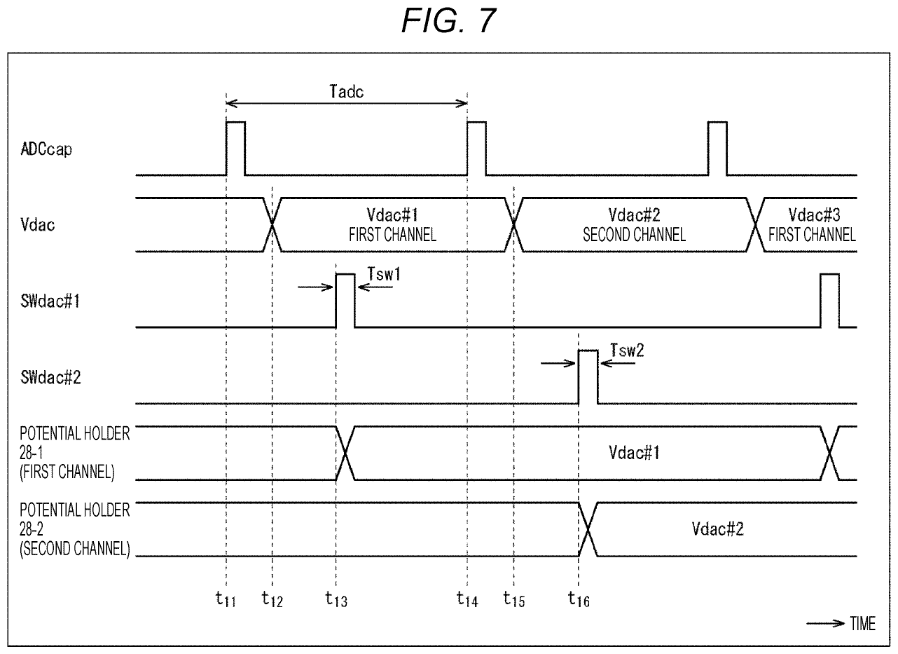

FIG. 7 is a diagram illustrating a timing chart regarding switching of the offset potential.

As illustrated in an uppermost part of FIG. 7, the AD conversion timing signal ADCcap becomes “H” at a cycle according to the count value Tadc.

In an example illustrated in FIG. 7, the AD conversion timing signal ADCcap becomes “H” at a time t₁₁, and as illustrated in a second part, the offset setting value Vdac according to the digital signal value Vadc of a reception signal of the first channel is updated from a certain offset setting value to the offset setting value Vdac #1 at a time t₁₂. The DA converter 26 performs DA conversion on a digital signal according to the offset setting value Vdac #1 and outputs a signal obtained through the DA conversion.

As illustrated in a third part, at a time t₁₃ after the offset setting value Vdac of the first channel is updated, the switching signal SWdac #1 becomes “H”, and the switch 27-1 turns on. In addition, as illustrated in a fifth part, immediately after the switch 27-1 turns on, the potential held by the potential holder 28-1 is updated to the value Vdac #1.

After the offset potential of the first channel held by the potential holder 28-1 is updated, the switch 27-1 turns off. The switching signal SWdac #1 becomes “H” for a period of time Tsw1. Thereafter, the update of the offset potential of the second channel is started.

As illustrated in the uppermost part, the AD conversion timing signal ADCcap becomes “H” at a time t14, and as illustrated in the second part, the offset setting value Vdac according to the digital signal value Vadc of a reception signal of the second channel is updated from a certain offset setting value to the offset setting value Vdac #2 at a time tis. The DA converter 26 performs DA conversion on a digital signal according to the offset setting value Vdac #2 and outputs a signal obtained through the DA conversion.

As illustrated in a fourth part, at a time tis after the offset setting value Vdac of the second channel is updated, the switching signal SWdac #2 becomes “H”, and the switch 27-2 turns on. In addition, as illustrated in a sixth part, immediately after the switch 27-2 turns on, the potential held by the potential holder 28-2 is updated to the value Vdac #2.

After the offset potential of the second channel held by the potential holder 28-2 is updated, the switch 27-2 turns off. The switching signal SWdac #2 becomes “H” for a period of time Tsw2. Thereafter, the update of the offset set value Vdac of the first channel and the update of the offset set value Vdac of the second channel are sequentially performed.

As described above, the control of the switch 27-1 and the switch 27-2 is performed in a period different from the period in which the digital signal value is obtained. Even in a case where signals input to the signal reception unit 13 are signals of a plurality of channels, the offset potential is updated for the AD converter 25 in a state where the AD converter 25 and the DA converter 26 are separated from each other, so that it is possible to prevent noise caused by the DA converter 26 from affecting a result of the AD conversion performed by the AD converter 25.

For example, in a case where attention is paid to the first channel, the switch 27-1 is controlled to update the offset set value Vdac of the first channel at a cycle twice as long as a cycle according to the value Tadc, which is a cycle for obtaining the digital signal value with the AD conversion timing signal ADCcap set to “H”. For example, in a case where attention is paid to the second channel, the switch 27-2 is controlled to update the offset set value Vdac of the second channel at a cycle twice as long as the cycle according to the value Tadc, which is the cycle for obtaining the digital signal value with the AD conversion timing signal ADCcap set to “H”.

As described above, a control cycle of a switch corresponding to one channel is variable in accordance with the number of channels of a biopotential signal. In the example described with reference to FIG. 5 (an example where there is one channel of a biopotential signal), the switch 27 is controlled to update the offset set value Vdac at the same cycle as that for obtaining the digital signal value with the AD conversion timing signal ADCcap set to “H”.

The switch 27-1 and the switch 27-2 may be controlled so as not to be turned on at different timings but to be turned on at the same time. In this case, the control timing determination unit 33 synchronously controls the switch 27-1 and the switch 27-2. The update of the potential held by the potential holder 28-1 and the update of the potential held by the potential holder 28-2 are performed in synchronization.

### <5. Others>

The signal input to the signal reception unit 13 may be signals of three or more channels.

The effects described herein are merely examples and are not restrictive, and other effects may also be produced.

An embodiment of the present technology is not limited to the above-described embodiment, and various modifications can be made without departing from the scope of the present technology.

In addition, each step described in the above flowchart can be executed by one device or shared and performed by a plurality of devices.

Moreover, in a case where a plurality of processing steps is included in one step, the plurality of processing included in the one step can be performed by one device or shared and performed by a plurality of devices.

### EXAMPLES OF COMBINATIONS OF CONFIGURATIONS

The present technology may also have the following configurations.

(1)

A biopotential measurement device including:
an amplifier that amplifies a potential that is a difference between a reference potential and a potential measured by an electrode attached to a measurement target position of a biopotential;
an AD converter that converts an analog signal according to the potential amplified by the amplifier into a digital signal;
a calculation unit that sets a setting value of an offset potential to be applied to an input of the amplifier on a basis of a signal level of the digital signal after AD conversion;
a DA converter that converts a digital signal indicating the setting value into an analog signal and that generates an analog potential;
a potential holder that holds the analog potential generated by the DA converter and that applies the held analog potential to the input of the amplifier;
a switch provided between the DA converter and the potential holder; and
a controller that switches a connected state and a separated state of the switch.
   (2)

The biopotential measurement device according to (1), further including:
a threshold determiner that compares the signal level of the digital signal after the AD conversion with a threshold,
in which the calculation unit sets the setting value according to a result of the comparison between the signal level and the threshold.
   (3)

The biopotential measurement device according to (2), further including:
a threshold holder that holds the threshold.
   (4)

The biopotential measurement device according to any one of (1) to (3), further including:
a reception signal obtainer that obtains a digital signal value on a basis of the digital signal after the AD conversion.
   (5)

The biopotential measurement device according to (4),
in which the controller controls the switch such that the switch enters the connected state in a period different from a period in which the digital signal value is obtained.
   (6)

The biopotential measurement device according to (5),
in which the controller controls the switch such that the switch enters the separated state after the analog potential held by the potential holder is updated.
   (7)
The biopotential measurement device according to any one of (1) to (6),
in which the AD converter outputs the digital signal after the AD conversion at a certain cycle in accordance with a control signal supplied from the controller.
   (8)

The biopotential measurement device according to (7),
in which the controller controls the switch at the certain cycle.
   (9)

The biopotential measurement device according to (8),
in which the cycle at which the switch is controlled is variable.
   (10)

The biopotential measurement device according to any one of (1) to (9),
in which the measurement target position includes a plurality of measurement target positions, and
a plurality of the amplifier, a plurality of the potential holder, and a plurality of the switch are provided in correspondence with to signals of results of measurement of the biological potentials.
   (11)

The biopotential measurement device according to (4),
in which the controller controls each of the plurality of switches such that the switch enters the connected state in a period different from a period in which the digital signal value is obtained.
   (12)

The biopotential measurement device according to (11),
in which the controller controls the plurality of switches such that the plurality of switches enters the connected state in different periods.
   (13)

The biopotential measurement device according to (11) or (12),
in which the controller controls the plurality of switches in synchronization.
   (14)

A biopotential measurement method used by a biopotential measurement device including
an amplifier that amplifies a potential that is a difference between a reference potential and a potential measured by an electrode attached to a measurement target position of a biopotential,
an AD converter that converts an analog signal according to the potential amplified by the amplifier into a digital signal,
a DA converter that converts a digital signal indicating a setting value of an offset potential to be applied to an input of the amplifier into an analog signal and that generates an analog potential,
a potential holder that holds the analog potential generated by the DA converter and that applies the held analog potential to the input of the amplifier, and
a switch provided between the DA converter and the potential holder,
the biological measurement method including:
setting the setting value on a basis of a signal level of the digital signal after the AD conversion; and
switching a connected state and a separated state of the switch.
   (15)

An information processing apparatus including:
a biopotential measurement device including
an amplifier that amplifies a potential that is a difference between a reference potential and a potential measured by an electrode attached to a measurement target position of a biopotential,
an AD converter that converts an analog signal according to the potential amplified by the amplifier into a digital signal,
a calculation unit that sets a setting value of an offset potential to be applied to an input of the amplifier on a basis of a signal level of the digital signal after the AD conversion,
a DA converter that converts a digital signal indicating the setting value into an analog signal and that generates an analog potential,
a potential holder that holds the analog potential generated by the DA converter and that applies the held analog potential to the input of the amplifier,
a switch provided between the DA converter and the potential holder, and
a controller that switches a connected state and a separated state of the switch.

### REFERENCE SIGNS LIST

1 Biosensor module
24 Differential amplifier
25 AD converter
26 DA converter
27 Switch
28 Potential holder
32 Offset setting value calculation unit
33 Control timing determination unit
43 Reception signal threshold determination section

1. A biopotential measurement device comprising:
an amplifier that amplifies a potential that is a difference between a reference potential and a potential measured by an electrode attached to a measurement target position of a biopotential;
an AD converter that converts an analog signal according to the potential amplified by the amplifier into a digital signal;
a calculation unit that sets a setting value of an offset potential to be applied to an input of the amplifier on a basis of a signal level of the digital signal after the AD conversion;
a DA converter that converts a digital signal indicating the setting value into an analog signal and that generates an analog potential;
a potential holder that holds the analog potential generated by the DA converter and that applies the held analog potential to the input of the amplifier;
a switch provided between the DA converter and the potential holder; and
a controller that switches a connected state and a separated state of the switch.

2. The biopotential measurement device according to claim 1, further comprising:
a threshold determiner that compares the signal level of the digital signal after the AD conversion with a threshold,
wherein the calculation unit sets the setting value according to a result of the comparison between the signal level and the threshold.

3. The biopotential measurement device according to claim 2, further comprising:
a threshold holder that holds the threshold.

4. The biopotential measurement device according to claim 1, further comprising:
a reception signal obtainer that obtains a digital signal value on a basis of the digital signal after the AD conversion.

5. The biopotential measurement device according to claim 4,
wherein the controller controls the switch such that the switch enters the connected state in a period different from a period in which the digital signal value is obtained.

6. The biopotential measurement device according to claim 5,
wherein the controller controls the switch such that the switch enters the separated state after the analog potential held by the potential holder is updated.

7. The biopotential measurement device according to claim 1,
wherein the AD converter outputs the digital signal after the AD conversion at a certain cycle in accordance with a control signal supplied from the controller.

8. The biopotential measurement device according to claim 7,
wherein the controller controls the switch at the certain cycle.

9. The biopotential measurement device according to claim 8,
wherein the cycle at which the switch is controlled is variable.

10. The biopotential measurement device according to claim 1,
wherein the measurement target position includes a plurality of measurement target positions, and
a plurality of the amplifier, a plurality of the potential holder, and a plurality of the switch are provided in correspondence with to signals of results of measurement of the biological potentials.

11. The biopotential measurement device according to claim 4,
wherein the controller controls each of the plurality of switches such that the switch enters the connected state in a period different from a period in which the digital signal value is obtained.

12. The biopotential measurement device according to claim 11,
wherein the controller controls the plurality of switches such that the plurality of switches enters the connected state in different periods.

13. The biopotential measurement device according to claim 11,
wherein the controller controls the plurality of switches in synchronization.

14. A biopotential measurement method used by a biopotential measurement device including
an amplifier that amplifies a potential that is a difference between a reference potential and a potential measured by an electrode attached to a measurement target position of a biopotential,
an AD converter that converts an analog signal according to the potential amplified by the amplifier into a digital signal,
a DA converter that converts a digital signal indicating a setting value of an offset potential to be applied to an input of the amplifier into an analog signal and that generates an analog potential,
a potential holder that holds the analog potential generated by the DA converter and that applies the held analog potential to the input of the amplifier, and
a switch provided between the DA converter and the potential holder,
the biological measurement method comprising:
setting the setting value on a basis of a signal level of the digital signal after the AD conversion; and
switching a connected state and a separated state of the switch.

15. An information processing apparatus comprising:
a biopotential measurement device including
an amplifier that amplifies a potential that is a difference between a reference potential and a potential measured by an electrode attached to a measurement target position of a biopotential,
an AD converter that converts an analog signal according to the potential amplified by the amplifier into a digital signal,
a calculation unit that sets a setting value of an offset potential to be applied to an input of the amplifier on a basis of a signal level of the digital signal after the AD conversion,
a DA converter that converts a digital signal indicating the setting value into an analog signal and that generates an analog potential,
a potential holder that holds the analog potential generated by the DA converter and that applies the held analog potential to the input of the amplifier,
a switch provided between the DA converter and the potential holder, and
a controller that switches a connected state and a separated state of the switch.
